(19) United States
(12) Patent Application Publication
(10) Pub. No.: US 2011/0313179 A1
(43) Pub. Date: Dec. 22, 2011

(54) METHOD FOR PRODUCING OLEFIN OXIDE

(75) Inventors: Tomonori Kawabata, Toyonaka-shi (JP); Makoto Yako, Wakayama-shi (JP)

(73) Assignee: SUMITOMO CHEMICAL COMPANY, LIMITED, Chuo-ku, Tokyo (JP)

(21) Appl. No.: 13/202,399

(22) PCT Filed: Mar. 12, 2010
(86) PCT No.: PCT/JP2010/054718

§ 371 Date: Aug. 19, 2011

(30) Foreign Application Priority Data
Mar. 13, 2009 (JP) 2009-060940

(51) Int. Cl. C07D 301/12

(52) U.S. Cl. 549/531

(57) ABSTRACT

The invention provides a method for producing an olefin oxide which comprises reacting hydrogen peroxide and an olefin in a solvent containing water, a nitrile compound and a cyclic secondary amine compound, in the presence of a titanosilicate having a pore composed of a 12- or more-membered oxygen ring.

### TECHNICAL FIELD

The present invention relates to a method for producing an olefin oxide.

### BACKGROUND ART

As a method for producing an olefin oxide, a method which employs a zeolite compound as the catalyst is known. JP 2001-97965 A describes a method of producing propylene oxide from hydrogen peroxide and propylene in a mixed solvent of water and methanol, in the presence of n-propylamine and a zeolite compound [TS-1] which has an MF1 structure.

### SUMMARY OF INVENTION

The present application relates to the following invention:

[1] A method for producing an olefin oxide which comprises reacting hydrogen peroxide and an olefin in a solvent containing water, a nitrile compound and a cyclic secondary amine compound, in the presence of a titanosilicate having a pore composed of a 12- or more-membered oxygen ring.
[2] The method according to [1], wherein the nitrile compound is acetonitrile.
[3] The method according to [1], wherein the cyclic secondary amine compound is piperidine or hexamethyleneimine.
[4] The method according to [1], wherein the titanosilicate has an X-ray diffraction pattern reproduced characterized by interplanar spacings d of
1.24±0.08 nm,
1.08±0.03 nm,
0.90±0.03 nm,
0.60±0.03 nm,
0.39±0.01 nm, and
0.34±0.01 nm.
   [5] The method according to [1] above, wherein the titanosilicate is a titanosilicate having an MWW structure, or a Ti-MWW precursor.
   [6] The method according to [1], wherein hydrogen peroxide is synthesized during reacting hydrogen peroxide and an olefin, in the solvent where the hydrogen peroxide and the olefin are reacted.
   [7] The method according to [1], wherein the olefin is propylene.

### DESCRIPTION OF EMBODIMENTS

According to the production method of the invention, an olefin oxide is synthesized from hydrogen peroxide and an olefin. The olefin oxide synthesis of the invention is carried out in the presence of the titanosilicate described below.

A titanosilicate is a generic name for a silicate having a four-coordinate Ti (titanium atom), which has a porous structure. In the present invention, a titanosilicate means a titanosilicate having substantially four-coordination Ti, which shows the maximum absorption peak in the wavelength range of 210 nm to 230 nm in the ultraviolet-visible absorption spectra in the wavelength range of 200 nm to 400 nm (for example, FIGS. 2(d) and (e) in Chemical Communications 1026-1027, (2002)). The ultraviolet visible absorption spectra can be measured by a diffuse reflectance method using an ultraviolet-visible spectrophotometer equipped with a diffuse reflection apparatus.

The titanosilicate in the present invention has a pore composed of a 12- or more-membered oxygen ring.

In the present description, a pore means a pore composed of a Si—O bond or a Ti—O bond. The pore may be a hemispherical pore, which is referred to as a side pocket. In other words, the pore is not required to penetrate through primary particles of the titanosilicate.

The “12- or more-membered oxygen ring” means a ring structure which has 12 or more oxygen atoms at (a) the cross-section of the narrowest portion of a pore or (b) the pore opening.

The pore has a pore size of generally 0.6 nm to 1.0 nm.

According to the invention, the pore size is the diameter of (a) a cross-section at the narrowest part of the pore or (b) the entrance of the pore.

The pore, pore size and interlayer distance for the titanosilicate are usually confirmed by analysis of the X-ray diffraction pattern. If the structure is known, it can be easily confirmed by comparison with the X-ray diffraction pattern.

Examples of the titanosilicate according to the present invention include those having the following structures represented by the structural code specified by the International Zeolite Association (IZA).

Ti-ZSM-12 having an MTW structure (for example, one described in Zeolites 15, 236-242 (1995)),

Ti-Beta having a BEA structure (for example, one described in Journal of Catalysis 199, 41-47 (2001)),

Ti-MOR having an MOR structure (for example, one described in The Journal of Physical Chemistry B 102, 9297-9303 (1998)),

Ti-ITQ-7 having an ISV structure (for example, one described in Chemical Communications 761-762 (2000)),

Ti-MCM-68 having an MSE structure (for example, one described in Chemical Communications 6224-6226 (2008)),

Ti-MWW having an MWW structure (for example, one described in Chemistry Letters 774-775 (2000)),

Ti-UTD-1 having a DON structure (for example, one described in Zeolites 15, 519-525 (1995)),

Ti-MWW precursors (for example, one described in JP 2005-262164 A),

Ti-MCM-36 (for example, one described in Catalysis Letters 113, 160-164 (2007)),

Ti-MCM-56 (for example, one described in Microporous and Mesoporous Materials 113, 435-444 (2008)),

Ti-YNU-1 (for example, one described in Angewandte Chemie International Edition 43 236-240 (2004)),

Ti-MCM-41 (for example, one described in Microporous Materials 10, 259-271 (1997)),

Ti-MCM-48 (for example, one described in Chemical Communications 145-146 (1996)) and

Ti-SBA-15 (for example, one described in Chemistry of Materials 14, 1657-1664 (2002)).

Of the titanosilicates mentioned above, Ti-MWW precursor and Ti-YNU-1 respectively have a laminar structure an interlayer space of which is different from an MWW structure. In the Ti-MWW precursor or Ti-YNU-1, an interlayer distance is generally 0.2 to 2 nm.

The Ti-MWW precursor means a titanosilicate which is converted into Ti-MWW by dehydrating condensation. The fact that the Ti-MWW precursor has a pore composed of a 12- or more-membered oxygen ring can be easily confirmed from the structure of the corresponding Ti-MWW.

According to the invention, the titanosilicate preferably has the following X-ray diffraction pattern:

Interplanar Spacing d
1.24±0.08 nm (12.4±0.8 Å)
1.08±0.03 nm (10.8±0.3 Å)
0.90±0.03 nm (9.0±0.3 Å)
0.60±0.03 nm (6.0±0.3 Å)
0.39±0.01 nm (3.9±0.1 Å)
0.34±0.01 nm (3.4±0.1 Å).

The X-ray diffraction pattern can be measured using an X-ray diffraction apparatus with copper K-alpha radiation. Examples of titanosilicate with this X-ray diffraction pattern include a Ti-MWW precursor, Ti-YNU-1, Ti-MWW and Ti-MCM-68.

The titanosilicate of the invention can be synthesized, for example, by any method described in the literature cited above. The titanosilicate may contain some of the structure-directing agents used for the synthesis of it. The structure-directing agent is a compound that contributes to formation of the titanosilicate structure. Examples of such structure-directing agents include nitrogen-containing organic compounds, including amine compounds such as piperidine and hexamethyleneimine. According to the invention, the structure-directing agent may be the same kind of compound as the cyclic secondary amine. The structure-directing agent is distinguished from a cyclic secondary amine in that the former is used for synthesis of a titanosilicate while the latter does not contribute to the synthesis.

According to the invention, the titanosilicate is more preferably a Ti-MWW precursor, and even more preferably a Ti-MWW precursor with a silicon/nitrogen-containing ratio (Si/N ratio) of 8 to 35. In the present description, the Si/N ratio is the value determined by elemental analysis. A Ti-MWW precursor having such a Si/N ratio contains a greater amount of structure-directing agent than an ordinary Ti-MWW precursor.

The Ti-MWW precursor can be prepared, for example, by any of the following methods 1-4.

1. A method comprising a hydrothermal synthesis step in which a boron compound, a titanium compound, a silicon compound, a structure-directing agent and water are mixed and then the mixture as obtained is heated, and a step in which a laminar compound obtained by the hydrothermal synthesis step (also called an as-synthesized sample) is contacted with an aqueous strong acid under reflux conditions (for example, JP 2005-262164 A). In this method, the titanium compound may be dissolved in the aqueous strong acid if necessary.
2. A method comprising a step in which a borosilicate with an MWW structure [B-MWW] is prepared, and a laminar compound is formed from the B-MWW, followed by contacting the laminar compound with 6 M nitric acid (for example, Chemical Communication 1026-1027 (2002),).
3. A method comprising a first step in which a structure-directing agent, a boron compound, a silicon compound and water are mixed and then the mixture as obtained is heated, and a second step in which the laminar compound obtained from the first step is contacted with a titanium compound and an inorganic acid.
4. A method in which a mixture of Ti-MWW, piperidine and water is heated to obtain a Ti-MWW precursor (for example, Catalysis Today 117 (2006) 199-205,).

In each of these methods, the structure-directing agent may be piperidine. Examples of the aqueous strong acid solution include nitric acid. According to Catalysis Today 117 (2006) 199-205, a Ti-MWW precursor obtained by method 4 has a Si/N ratio of 8.5 to 8.6, and a higher nitrogen content than conventionally known Ti-MWW precursors. According to the invention, the Ti-MWW precursor is preferably one obtained by method 4.

The titanosilicate used for the invention may also be silylated using a silylating agent such as 1,1,1,3,3,3-hexamethyldisilazane. Silylation of the titanosilicate is preferred for greater activity and higher selectivity.

The titanosilicate may also be further activated by treatment involving contact with a hydrogen peroxide solution. The hydrogen peroxide concentration is generally in the range of 0.0001 wt % to 50 wt %. While there are no particular restrictions on the solvent of the hydrogen peroxide solution, it is preferred, because of convenience to the industrial use, to use water or a solvent ordinarily used for propylene oxide synthesis reaction.

According to the invention, the amount of titanosilicate may be appropriately selected depending on its kinds and on the reaction scale. The lower limit of the amount is usually 0.01 part by weight, preferably 0.1 part by weight and more preferably 0.5 part by weight, with respect to 100 parts by weight of the solvent. The upper limit of the amount is usually 20 parts by weight, preferably 10 parts by weight and more preferably 8 parts by weight, with respect to 100 parts by weight of the solvent.

The process of the present invention is carried out in a solvent containing water, a nitrile compound and a cyclic secondary amine compound.

Examples of the cyclic secondary amine compound include the compounds of formula (I).

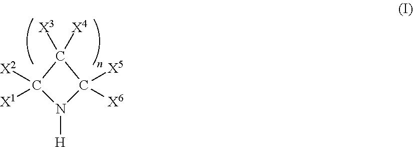

(In formula (I), X¹, X², X³, X⁴, X⁵ and X⁶ independently represent hydrogen, optionally substituted alkyl, optionally substituted alkenyl, optionally substituted alkynyl, optionally substituted cycloalkyl or optionally substituted aryl, where groups among X¹, X², X³, X⁴, X⁵ and X⁶, which are bonded to adjacent carbon atoms on the imine ring, are combined, together with adjacent carbon atoms, to represent a fused ring, and n represents an integer of 0-5.)

The alkyl, alkenyl, alkynyl, cycloalkyl and aryl groups in formula (I) will now be explained. Examples of alkyl groups include alkyl groups having 1 to 6 carbon atoms such as methyl, ethyl, propyl, butyl, pentyl and hexyl. Examples of alkenyl groups include alkenyl groups having 2 to 6 carbon atoms such as vinyl, propenyl, butenyl, pentenyl and hexenyl. Examples of alkynyl groups include ethynyl, propynyl, butynyl, pentynyl and hexynyl. Examples of cycloalkyl groups include cycloalkyl groups having 3 to 6 carbon atoms such as cyclopropyl, cyclobutyl, cyclopentyl and cyclohexyl.

Examples of aryl groups include phenyl and naphthyl.

The alkyl, alkenyl and alkynyl groups may be optionally substituted with one or more substituents selected from Group A defined below. The cycloalkyl group may be optionally substituted with one or more substituents selected from Group B defined below. The aryl group may be optionally substituted with one or more substituents selected from Group C defined below.

Group A: The group consisting of cycloalkyl, aryl, alkoxy, formyl, carboxyl, alkoxycarbonyl, hydroxyl, mercapto, halogens and amino.
Group B: The group consisting of alkyl, alkenyl, alkynyl, cycloalkyl, aryl, alkoxy, formyl, carboxyl, alkoxycarbonyl, hydroxyl, mercapto, halogens and amino.
Group C: The group consisting of alkyl, alkenyl, alkynyl, cycloalkyl, alkoxy, formyl, carboxyl, alkoxycarbonyl, hydroxyl, mercapto, halogens and amino.

Examples of alkoxy groups include alkoxy groups having 1 to 6 carbon atoms such as methoxy, ethoxy, propoxy, butoxy, pentyloxy and hexyloxy. Examples of alkoxycarbonyl groups include alkoxycarbonyl groups having 2 to 7 carbon atoms such as methoxycarbonyl, ethoxycarbonyl, propoxycarbonyl, butoxycarbonyl, pentyloxycarbonyl and hexyloxycarbonyl. The cyclic secondary amine compound may be a compound of formula (I) wherein one or more of —CX¹(X²)—, —CX³(X⁴)— and —CX⁵(X⁶)— has been converted to —C(═O)—.

Specific examples of the cyclic secondary amine compound include aziridine, azetidine, pyrrolidine, piperidine, hexamethyleneimine, azocane, ε-caprolactam, δ-valerolactam, ω-laurinelactam, 2,2,6,6-tetramethylpiperidine, 2-pipecoline, 3-pipecoline, 4-pipecoline, 1,2,3,4-tetrahydroquinaldine, 1,2,3,4-tetrahydroquinoline, 4-piperidine ethanol, 3-piperidineethanol, 4-piperidinemethanol, 2-methylpiperidine, 4-methylpiperidine, 1,2,2,6,6-pentamethylpiperidine, 3,5-dimethylpiperidine, 2,6-dimethylpiperidine, 2-ethylpiperidine, 4-ethylpiperidine, 1,2,2,6,6-pentaethylpiperidine, 3,5-diethylpiperidine and 2,6-diethylpiperidine.

Examples of the cyclic secondary amine compounds preferably include compounds of formula (I) wherein n=2-6 and X¹, X², X³, X⁴, X⁵ and X⁶ are hydrogen or alkyl groups having 1 to 6 carbon atoms, and more preferably include piperidine and hexamethyleneimine.

According to the invention, a salt of a cyclic secondary amine compound may also be used. Such salts may be inorganic acid or organic acid salts of the cyclic secondary amine compounds mentioned above.

The acid may be hydrochloric acid, sulfuric acid, nitric acid, phosphoric acid, carbonic acid, a fatty acid having 2 to 12 carbon atoms, an alkylsulfuric acid having 1 to 3 carbon atoms or sulfonic acid. Preferred salts of cyclic secondary amine compounds include acid salts of piperidine or hexamethyleneimine.

According to the invention, the amount of cyclic secondary amine compound used is usually in the range of 0.1 ppm by weight to 5000 ppm by weight and preferably in the range of 1 ppm by weight to 1000 ppm by weight, more preferably in the range of 10 ppm by weight to 200 ppm by weight, still more preferably in the range of 10 ppm by weight to 100 ppm by weight with respect to the solvent weight. The reaction selectivity and high titanosilicate activity will be high so long as the amount of cyclic secondary amine compound is within this range. When a salt of a cyclic secondary amine compound is used as the cyclic secondary amine compound, the amount of the salt may be such that the amount corresponding to the cyclic secondary amine compound moiety in the salt is within the range specified above.

According to the invention, propylene oxide is synthesized in a solvent comprising water and a nitrile compound. The nitrile compound may be a linear or branched saturated aliphatic nitrile or an aromatic nitrile. Specific examples of the nitrile compound include alkylnitriles having 2 to 4 carbon atoms such as acetonitrile, propionitrile, isobutyronitrile or butyronitrile and benzonitriles having 6 to 10 carbon atoms, with acetonitrile being preferred. The proportion of the water and nitrile compound (=water:nitrile compound) is usually 90:10 to 0.01:99.99, preferably 50:50 to 0.1:99.9 and more preferably 40:60 to 5:95 by weight.

The propylene oxide is synthesized from an olefin and hydrogen peroxide in the manner described above. The olefin may be a linear or branched olefin having 2 to 10 carbon atoms or a cyclic olefin having 4 to 10 carbon atoms. Examples of linear or branched olefins include ethylene, propylene, butene, pentene, hexene, heptene, octene, nonene, decene, 2-butene, isobutene, 2-pentene, 3-pentene, 2-hexene, 3-hexene, 4-methyl-1-pentene, 2-heptene, 3-heptene, 2-octene, 3-octene, 2-nonene, 3-nonene, 2-decene and 3-decene. Examples of the cycloolefin include cyclobutene, cyclopentene, cyclohexene, cycloheptene, cyclooctene, cyclononene and cyclodecene. The olefin is preferably a linear or branched olefin having 2 to 6 carbon atoms, more preferably propylene.

The hydrogen peroxide used may be a 0.0001 wt % to 100 wt % aqueous hydrogen peroxide solution, or hydrogen peroxide itself. According to the invention, the amount of hydrogen peroxide used is not particularly restricted, which is usually in the range of olefin:hydrogen peroxide (molar ratio)=1000:1 to 1:1000 and preferably 100:1 to 1:100.

The hydrogen peroxide may be prepared by a known process. The hydrogen peroxide may also be produced from oxygen and hydrogen during reacting hydrogen peroxide and an olefin, in the solvent where the hydrogen peroxide and the olefin are reacted. A noble metal is generally used as the catalyst when hydrogen peroxide is produced from oxygen and hydrogen. Examples of the noble metal include palladium, platinum, ruthenium, rhodium, iridium, osmium and gold, as well as alloys of the same. Preferred noble metal is palladium, platinum or gold. Palladium is even more preferred as the noble metal. The palladium may be palladium colloid (for example, Example 1 in JP 2002-294301 A).

A single noble metal or its alloy may be used, or a combination of two or more may be used together. For example, when palladium is used as the noble metal, it may be used with a metal other than palladium, such as platinum, gold, rhodium, iridium or osmium. Preferred metals other than palladium include gold and platinum.

The noble metal may also be used as a noble metal compound, such as an oxide or hydroxide. The noble metal compound is generally reduced by the copresent hydrogen to be converted to the noble metal.

The noble metals are preferably used in the form of being supported on a carrier. Examples of a carrier include the aforementioned titanosilicates; oxides such as silica, alumina, titania, zirconia and niobia; hydroxides of niobic acid, zirconic acid, tungstic acid or titanic acid; carbon; and mixtures of the foregoing. The carrier is preferably carbon. Active carbon, carbon black, graphite and carbon nanotubes are known forms of carbon for carriers.

The weight ratio of the noble metal with respect to the titanosilicate (noble metal weight/titanosilicate weight) is preferably 0.01/100 to 100/100 and more preferably 0.1/100 to 20/100.

The method for supporting the noble metal on the carrier may be a known method, for example, a method which comprises supporting the noble metal compound on the carrier by impregnation and then reducing the compound.

The reduction process may be reduction with a reducing agent such as hydrogen. When the noble metal compound is a noble metal amine complex, it will be reduced by the ammonia gas generated by its thermal decomposition in an inert gas.

The reduction temperature differs depending on the kind of noble metal compound used. For example in the case that Pd tetramine chloride is used as the noble metal compound, the temperature is usually between 100° C. and 500° C. and preferably between 200° C. and 350° C.

The carrier comprising the noble metal comprises the noble metal in the range of generally 0.01-20 wt %, and preferably 0.1-5 wt %.
The noble metal may be used as a mixed catalyst, obtained by mixing of the noble metal-supported carrier with titanosilicate.

Addition of a buffer salt to the solvent in the method for producing olefin oxide can further increase reaction efficiency. The amount of buffer salt added is usually 0.001 mmol to 100 mmol with respect to 1 kg of solvent.

Examples of buffer salts include buffer salts comprising 1) an anion selected from among sulfate ion, hydrogensulfate ion, carbonate ion, hydrogencarbonate ion, phosphate ion, hydrogenphosphate ion, dihydrogenphosphate ion, hydrogenpyrophosphate ion, pyrophosphate ion, halogen ions, nitrate ion, hydroxide ion and carboxylate ions having 1 to 10 carbon atoms, and 2) a cation selected from among ammonium, alkylammonium, alkylarylammonium, alkali metal cations and alkaline earth metal salt cations.

Examples of carboxylate ions having 1 to 10 carbon atoms include acetate ion, formate ion, acetate ion, propionate ion, butyrate ion, valerate ion, caproate ion, caprylate ion, caprate ion and benzoate ion.

Examples of alkylammonium ions include tetramethylammonium, tetraethylammonium, tetra-n-propylammonium, tetra-n-butylammonium and cetyltrimethylammonium.

Examples of alkali metal cations or alkaline earth metal cations include lithium cation, sodium cation, potassium cation, rubidium cation, cesium cation, magnesium cation, calcium cation, strontium cation and barium cation.

Preferred buffer salts include ammonium salts of inorganic acids such as ammonium sulfate, ammonium hydrogensulfate, ammonium carbonate, ammonium hydrogencarbonate, diammonium hydrogenphosphate, ammonium dihydrogenphosphate, ammonium phosphate, ammonium hydrogenpyrophosphate, ammonium pyrophosphate, ammonium chloride and ammonium nitrate, and ammonium salts of carboxylic acids having 1 to 10 carbon atoms such as ammonium acetate, with ammonium dihydrogenphosphate being a preferred example of an ammonium salt.

A compound that generates a buffer salt ion, such as a noble metal amine complex, may also be used as the noble metal compound instead of the aforementioned buffer salt. For example, if Pd tetramine chloride is used as the noble metal compound and it is partially reduced, ammonium ion will be generated during synthesis of the olefin oxide.

A quinoid compound is preferably added to the solvent in the method for producing olefin oxide, to further increase the olefin oxide selectivity, when hydrogen peroxide is synthesized from oxygen and hydrogen during reacting hydrogen peroxide and an olefin, in the solvent where the hydrogen peroxide and the olefin are reacted.

Examples of quinoid compounds represent by the following formula (1).

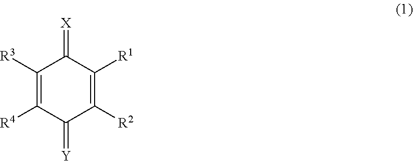

(In formula (1), R¹, R², R³ and R⁴ each independently represent hydrogen, or R¹ and R² are combined, together with their carbon atoms to which R¹ and R² are bonded, to represent an optionally substituted benzene ring, or R³ and R⁴ are combined, together with their carbon atoms to which R¹ and R² are bonded, to represent an optionally substituted benzene ring; and X and Y each independently represent an oxygen atom or NH group.)

Examples of compounds of formula (1) include:

1) a quinone compound (1A) represented by the formula (1) wherein R¹, R², R³, and R⁴ are a hydrogen atom, and both X and Y are an oxygen atom;
2) a quinoneimine compound (1B) represented by the formula (1) wherein R¹, R², R³, and R⁴ are a hydrogen atom, and X and Y are an oxygen atom and an NH group, respectively; and
3) a quinonediimine compound (1C) represented by the formula (1) wherein R¹, R², R³, and R⁴ are a hydrogen atom, and X and Y are NH groups.

The quinoid compounds of formula (1) include the following anthraquinone compounds (2).

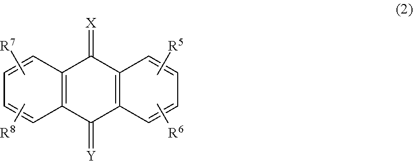

(In formula (2), X and Y are as defined in formula (1), and R⁵, R⁶, R⁷ and R⁸ each independently represent hydrogen, hydroxyl or alkyl (for example, an alkyl group having 1 to 5 carbon atoms such as methyl, ethyl, propyl, butyl or pentyl)).

In the formulas (1) and (2), X and Y preferably represent oxygen atoms.

Examples of quinoid compounds include benzoquinone, naphthoquinone, anthraquinone, alkylanthraquinone compound, polyhydroxyanthraquinone compounds.

Examples of alkylanthraquinone compounds include 2-alkylanthraquinone compounds such as 2-ethylanthraquinone, 2-t-butylanthraquinone, 2-amylanthraquinone, 2-methylanthraquinone, 2-butylanthraquinone, 2-t-amylanthraquinone, 2-isopropylanthraquinone, 2-s-butylanthraquinone and 2-s-amylanthraquinone; and polyalkylanthraquinone compounds such as 1,3-diethylanthraquinone, 2,3-dimethylanthraquinone, 1,4-dimethylanthraquinone and 2,7-dimethylanthraquinone. Examples of a polyhydroxyanthraquinone include 2,6-dihydroxyanthraquinone.

Preferred quinoid compounds include anthraquinone and 2-alkylanthraquinone compounds (compounds of formula (2) wherein X and Y are oxygen atoms, R⁵ is a 2-substituted alkyl group, R⁶ is a hydrogen atom and R⁷ and R⁸ are hydrogen atoms).

The amount of quinoid compound used can usually be in the range of 0.001 mmol/kg to 500 mmol/kg with respect to 1 kg of solvent. The preferred quinoid compound amount is 0.01 mmol/kg to 50 mmol/kg with respect to 1 kg of solvent.

The quinoid compound can be prepared by oxidation of the dihydro form of the quinoid compound with oxygen in a reaction system. For example, hydroquinone or a hydrogenated quinoid compound such as 9,10-anthracenediol is added to a solvent and oxidized by oxygen in the reactor to generate a quinoid compound.

Examples of dihydro forms of quinoid compounds include compounds of formulas (3) and (4), which are dihydro forms of the compounds of formulas (1) and (2).

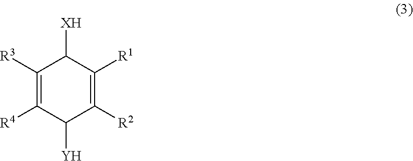

(In formula (3), R¹, R², R³, R⁴, X and Y are as defined in formula (1).)

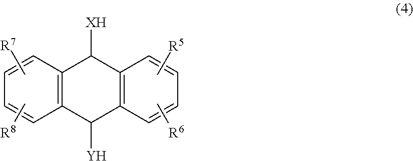

(In formula (4), X, Y, R⁵, R⁶, R⁷ and R⁸ are as defined in formula (2).)

X and Y in formula (3) and formula (4) preferably represent oxygen atoms.

Preferred dihydro forms of quinoid compounds are dihydro forms of the preferred quinoid compounds mentioned above.

The reaction method for olefin oxide synthesis may be a fixed-bed flow reaction, a slurry complete mixing flow reaction, or the like.

According to the invention, the amount of olefin supplied is appropriately selected depending on the kind and on the reaction scale, the lower limit of which is preferably 0.01 part by weight, more preferably 0.1 part by weight and especially 1 part by weight, with respect to 100 parts by weight as the total of the solvent. The upper limit of the amount is preferably 1000 parts by weight, more preferably 100 parts by weight and especially 50 parts by weight, with respect to 100 parts by weight as the total of the solvent.

When hydrogen peroxide is produced from oxygen and hydrogen, during the oxidation reaction, in the solvent where the oxidation reaction is performed, the partial pressure ratio of oxygen and hydrogen supplied to the reactor is generally in the range of 1:50 to 50:1.

The preferred oxygen and hydrogen partial pressure ratio is 1:2 to 10:1. If the oxygen and hydrogen partial pressure ratio (oxygen/hydrogen) is too high, the olefin oxide production rate may be reduced. If the oxygen and hydrogen partial pressure ratio (oxygen/hydrogen) is too low, the olefin oxide selectivity may be reduced due to increased alkane by-production.

The oxygen and hydrogen gas used for this reaction may be diluted with a diluting gas for the reaction. Diluting gases include nitrogen, argon, carbon dioxide, methane, ethane and propane. There are no particular restrictions on the density of the diluting gas, and the oxygen or hydrogen may be diluted as necessary for the reaction. The oxygen starting material may be oxygen gas, air or the like. The oxygen gas used may be oxygen gas produced by an inexpensive pressure swing process, or if necessary it may be high purity oxygen gas produced by cryogenic separation.

The reaction temperature for olefin oxide synthesis reaction is generally 0° C. to 200° C. and preferably 40° C. to 150° C. If the reaction temperature is too low the reaction rate may be reduced. If the reaction temperature is too high, by-products may be increased due to secondary reactions. The reaction pressure is not particularly restricted, which is usually 0.1 MPa to 20 MPa and preferably 1 MPa to 10 MPa as the gauge pressure. The reaction product can be obtained by distilling separation.

### EXAMPLES

Hereinafter, the present invention will be described with reference to Examples. However, the present invention is not intended to be limited to these Examples.

Analysis Methods for the Examples

### Elemental Analysis

The weights of Ti (titanium), Si (silicon) and B (boron) in the catalysts were determined by ICP emission spectroscopy. Specifically, approximately 20 mg of a sample was weighted into a platinum crucible and covered with sodium carbonate, followed by fusion procedures using a gas burner. After the fusion, the content in the platinum crucible was dissolved by heating in pure water and nitric acid. Then, the solution was diluted with pure water, and then each element in this measurement solution was quantified using an ICP emission spectroscope (ICPS-8000 manufactured by Shimadzu Corp.).

N (nitrogen) for a sample weighed out to 10-20 mg was measured with an oxygen circulating combustion/TCD detection system employing a SUMIGRAPH NCH-22F (product of Sumika Chemical Analysis Service, Ltd.) (reaction temperature: 850° C., reduction temperature: 600° C.). The separating column used was a porous polymer beads-filled column, with acetoanilide as the reference sample.

### X-Ray Powder Diffraction (XRD)

The X-ray powder diffraction pattern of the sample was measured with the following apparatus and conditions.

Apparatus: RINT2500 V manufactured by Rigaku Corp.
Source: Cu Kα-rays
Conditions: Output=40 kV-300 mA
Scan range: 20=0.75 to 20°
Scanning speed: 1°/min

The sample was identified as a Ti-MWW precursor if the X-ray diffraction pattern was similar to FIG. 1 in EP1731515A1.

The sample was identified as a titanosilicate with an MWW structure [Ti-MWW] if the X-ray diffraction pattern was similar to FIG. 2 in EP1731515A1.

### Ultraviolet-Visible Absorption Spectrum (UV-Vis)

The sample was thoroughly pulverized with an agate mortar and then pelletized (7 mmφ), and the ultraviolet-visible absorption spectrum was measured with the following apparatus and conditions.

Apparatus: Diffuse reflection sensor (Praying Mantis manufactured by HARRICK).
Attachment: Ultraviolet-visible spectrophotometer (V-7100 manufactured by JASCO Corp.)
Pressure: Atmospheric pressure
Measured value: Reflectance
Data capture time: 0.1 second
Band width: 2 nm
Measuring wavelength: 200 to 900 nm
Slit height: half-open
Data capture interval: 1 nm
Baseline compensation (reference): BaSO₄ pellets (7 mmφ)

Samples exhibiting a peak in the wavelength range of 210 nm to 230 nm in the ultraviolet and visible absorption spectrum were identified as titanosilicate.

### Gas Chromatography

A gas chromatograph HP5890 Series II, manufactured by Agilent Technologies) was used.

### Measurement of Hydrogen Peroxide Concentration

A 0.02 mol/L potassium permanganate solution was used with an automatic titration apparatus (COM-1600 manufactured by Hiranuma Sangyo Corp.) to determine the hydrogen peroxide concentration of the reaction mixture.

### Reference Example 1

A gel was prepared by stirring 899 g of piperidine, 2402 g of purified water, 112 g of tetra-n-butyl orthotitanate [TBOT], 565 g of boric acid and 410 g of fumed silica (cab-o-sil M7D) in an autoclave at room temperature under an air atmosphere. The gel was aged for 1.5 hours and then further heated to 160° C. over a period of 8 hours while stirring in a sealed state, after which it was stored at 160° C. for 96 hours to obtain a suspension. The obtained suspension was filtered, and a collected solid (i) was rinsed with water until the filtrate reached nearly pH 10.

The solid (i) was then dried at 50° C. until no further weight reduction was observed, to obtain 522 g of a solid (i-1). To 75 g of the solid (i-1) there was added 3750 mL of 2 M nitric acid, and the mixture was refluxed for 20 hours. The obtained reaction mixture was then filtered and the resulting solid (i-2) was rinsed with water to near neutral filtrate, and vacuum dried at 150° C. until no further weight reduction was observed, to obtain 60 g of a white powder.

The white powder was determined to be titanosilicate with an MWW precursor structure, based on its X-ray diffraction pattern and UV-visible absorption spectrum. The white powder had a Ti content of 1.67 wt %. This titanosilicate will hereunder be referred to as Ti-MWW precursor (A).

### Example 1

After placing 1.2 g of Ti-MWW precursor (A) in a 0.5 L volume autoclave, a continuous reaction in which nitrogen, propylene and a solution with the following composition were each supplied at the rates listed below and the reaction mixture was extracted from the autoclave through a filter, was carried out under conditions with a temperature of 60° C., a pressure of 3 MPa (gauge pressure) and a residence time of 15 minutes.

### Solution Composition

7 wt % H₂O₂, 85 ppm by weight piperidine, solvent: water/acetonitrile=20/80 (weight ratio)

### Supply Rates

Nitrogen: 500 mL/min
Propylene: 1401 mmol/hr
Water/acetonitrile solution: 380 mL/hr

### Example 2

The same procedure was carried out as in Example 1, except that the piperidine content in the water/acetonitrile solution was changed to 26 ppm by weight.

### Comparative Example 1

The same procedure was carried out as in Example 1, except that the piperidine in the water/nitrile solution was changed to 59 ppm by weight of n-propylamine.

### Comparative Example 2

The same procedure was carried out as in Example 1, except that 85 ppm by weight of piperidine was not added to the solution composition.

Table 1 shows the results of analyzing the liquid phases extracted after elapse of prescribed times for Examples 1-2 and Comparative Examples 1-2, using an automatic titrator.

TABLE 1

| Reaction time | Hydrogen peroxide conversion (%) | | | |
|---|---|---|---|---|
| (hr) | Example 1 | Example 2 | Comp. Ex. 1 | Comp. Ex. 2 |
| 1.5 | 96.6 | | | |
| 2 | | 88.7 | | 89.9 |
| 2.5 | 89.6 | | | |
| 3 | | 85.8 | 69.9 | 83.0 |
| 4 | 88.0 | 84.4 | 57.1 | 76.5 |
| 5 | 87.1 | 83.3 | 49.6 | 73.9 |
| 6 | 84.1 | | 45.4 | 70.5 |

### Reference Example 2

A gel was prepared by stirring 899 g of piperidine, 2402 g of purified water, 22.4 g of TBOT, 565 g of boric acid and 410 g of fumed silica (cab-o-sil M7D) in an autoclave at room temperature under an air atmosphere. The obtained gel was aged for 1.5 hours and further heated to 160° C. over a period of 8 hours while stirring in a sealed state, after which it was stored at 160° C. for 120 hours to obtain a suspension. The obtained suspension was filtered, and a collected solid (ii) was rinsed with water until the filtrate reached pH 10.4. The solid (ii) was then dried at 50° C. until no further weight reduction was observed, to obtain 564 g of a solid (ii-1).

To 75 g of the solid (ii-1) there was added 3750 mL of 2 M nitric acid and 9.5 g of TBOT, and the mixture was refluxed for 20 hours.

The obtained reaction mixture was then filtered, and the resulting solid (ii-2) was rinsed with water to a near neutral filtrate and vacuum dried at 150° C. until no further weight reduction was observed, to obtain 62 g of a white powder.

As a result of measurement of the X-ray diffraction pattern and ultraviolet and visible absorption spectrum, the white powder was confirmed to be a Ti-MWW precursor (this Ti-MWW precursor will hereunder be referred to as “Ti-MWW precursor (B)”). The Ti-MWW precursor (B) had a Ti content of 1.56 wt % and a Si/N ratio of 55.

A 60 g portion of the Ti-MWW precursor (B) was heated at 530° C. for 6 hours to obtain 54 g of a solid (ii-3). The solid (ii-3) was confirmed to be titanosilicate with an MWW structure [Ti-MWW], based on its X-ray diffraction pattern. The same procedure was carried out twice to obtain a total of 162 g of Ti-MWW.

A gel was prepared by dissolving 300 g of piperidine, 600 g of purified water and 110 g of the aforementioned Ti-MWW in an autoclave at room temperature under an air atmosphere while stirring, and after aging the gel for 1.5 hours, it was further heated to 160° C. over a period of 4 hours while stirring in a sealed state, after which it was stored at 160° C. for 24 hours to obtain a suspension. The obtained suspension was filtered, and a collected solid (ii-4) was rinsed with water until the filtrate reached nearly pH 9. The solid (ii-4) was then vacuum dried at 150° C. until no further weight reduction was observed, to obtain 108 g of a white powder.

As a result of measurement of the X-ray diffraction pattern and ultraviolet and visible absorption spectrum, the white powder was confirmed to be a titanosilicate with a Ti-MWW precursor structure (this Ti-MWW precursor will hereunder be referred to as “Ti-MWW precursor (C)”). The Ti-MWW precursor (C) had a Ti content of 1.58 wt % and a Si/N ratio of 10.

A 0.266 g portion of the Ti-MWW precursor (C) was contacted with 100 g of a 0.1 wt % hydrogen peroxide solution (solvent:water/acetonitrile=20/80 (weight ratio)) for 1 hour at room temperature. The Ti-MWW precursor (D) obtained by the contact was collected by filtration and rinsed with 500 mL of water.

### Reference Example 3

After washing 20 g of commercial active carbon (powder product of Wako Pure Chemical Industries, Ltd.) with 10 L of hot water (100° C.), it was dried at 150° C. under a nitrogen stream for 6 hours to obtain washed active carbon. The total amount of the washed active carbon was added to a 2 L volume volumetric flask containing 1 L of water, and the mixture was stirred under air at room temperature to obtain suspension (1). To suspension (1) there was slowly added dropwise 100 mL of an aqueous solution containing 6.12 g of Pd Colloid (product of JGC Catalysts and Chemicals, Ltd., Pd content: 3.0 wt %) under air at room temperature, to obtain suspension (2). Suspension (2) was stirred at room temperature for 8 hours under air. Upon completion of stirring, a rotary evaporator was used to remove the moisture, and then washing was performed with 5 L of hot water (100° C.), followed by 6 hours of drying at 150° C. under a nitrogen stream to obtain a noble metal catalyst.

### Example 3

After placing 0.266 g of Ti-MWW precursor (D) and 0.02 g of the noble metal catalyst in a 0.5 L volume autoclave, a source gas and solution with the compositions shown below were each supplied at the rates listed below, and continuous reaction, in which the reaction mixture was removed from the autoclave through a filter, was carried out under conditions with a temperature of 60° C., a pressure of 0.8 MPa (gauge pressure) and a residence time of 90 minutes.

### Source Gas

Composition: Propylene/oxygen/hydrogen/nitrogen (volume ratio)=6.5/4.5/11/78
Supply rate: 21.3NL/hr

### Starting Solution

Composition: 85 ppm by weight piperidine, 0.7 mmol/kg anthraquinone and 1 wt % propylene oxide, solvent: water/acetonitrile=20/80 (weight ratio).
Supply rate: 108 mL/hr

5 hours later from the beginning of the reaction, the liquid and gas phases were taken out from the reactor and analyzed by gas chromatography. The propylene oxide production volume was 10.88 mmol/hr and the propylene glycol selectivity was 1.1%.

### Example 4

The same procedure was carried out as in Example 3, except that 99 ppm by weight of hexamethyleneimine was used instead of 85 ppm by weight of piperidine. As a result of gas chromatography analysis of the liquid phase and gas phase that were extracted 5 hours after start of the reaction, the propylene oxide production volume was 10.14 mmol/hr and the propylene glycol selectivity was 1.8%.

### Comparative Example 3

The same procedure was carried out as in Example 3, except that 81 ppm by weight of ammonium dihydrogen phosphate was used instead of 85 ppm by weight of piperidine. 5 hours later from the beginning of the reaction, the liquid and gas phases were taken out from the reactor and analyzed by gas chromatography. The propylene oxide production volume was 9.28 mmol/hr and the propylene glycol selectivity was 4.4%.

### Comparative Example 4

The same procedure was carried out as in Example 3, except that 85 ppm by weight of piperidine was not used. 5 hours later from the beginning of the reaction, the liquid and gas phases were taken out from the reactor and analyzed by gas chromatography. The propylene oxide production volume was 8.86 mmol/hr and the propylene glycol selectivity was 4.8%.

### INDUSTRIAL APPLICABILITY

According to the method of the invention it is possible to efficiently produce olefin oxide.

1. A method for producing an olefin oxide which comprises reacting hydrogen peroxide and an olefin in a solvent containing water, a nitrile compound and a cyclic secondary amine compound, in the presence of a titanosilicate having a pore composed of a 12- or more-membered oxygen ring.

2. The method according to claim 1, wherein the nitrile compound is acetonitrile.

3. The method according to claim 1, wherein the cyclic secondary amine compound is piperidine or hexamethyleneimine.

4. The method according to claim 1, wherein the titanosilicate has an X-ray diffraction pattern characterized by interplanar spacings d of
1.24±0.08 nm,
1.08±0.03 nm,
0.90±0.03 nm,
0.60±0.03 nm,
0.39±0.01 nm, and
0.34±0.01 nm.

5. The method according to claim 1, wherein the titanosilicate is a titanosilicate having an MWW structure, or a Ti-MWW precursor.

6. The method according to claim 1, wherein hydrogen peroxide is synthesized during reacting hydrogen peroxide and an olefin, in the solvent where the hydrogen peroxide and the olefin are reacted.

7. The method according to claim 1, wherein the olefin is propylene.
